(19) United States
(12) Patent Application Publication
(10) Pub. No.: US 2016/0144123 A1
(43) Pub. Date: May 26, 2016

(54) Syringe adapter

(71) Applicant: John Berndt, Derry, NH (US)

(72) Inventor: John Berndt, Derry, NH (US)

(21) Appl. No.: 14/953,296

(22) Filed: Nov. 27, 2015

Related U.S. Application Data
(60) Provisional application No. 62/084,597, filed on Nov. 26, 2014.

(51) Int. Cl. A61M 5/315

(52) U.S. Cl. CPC A61M 5/31515

(57) ABSTRACT

A syringe adapter attaches to the syringe to prevent contamination of the fluid. After a withdrawal of sterile fluid in a syringe from a prescription bottle, the adapter may be attached to the syringe to prevent contamination of the fluid. The fluid may then be safely injected into the patient's body or used for bladder irrigation.

### CROSS-REFERENCE TO RELATED APPLICATIONS

This Application claims the benefit of U.S. Provisional Application No. 62/084,597, filed Nov. 26, 2014 which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to medical equipment and in particular to a syringe adaptor. During the course of medical care, it sometimes becomes necessary or advisable to withdraw a sterile fluid from a prescription bottle and, after a period of time, introduce the fluid into the patient's body. This may be injected via an intravenous catheter or used for bladder irrigation. Unfortunately, contamination of the fluid is fairly common, forcing a second withdrawal of fluid. This must be repeated as necessary until the fluid can be successfully transferred in a sterile condition. Also, when injecting via a catheter, a poorly fitting luer lock can cause backsplash of the fluid.

A search of the prior art reveals various syringes and syringe attachments which have been developed. None are closely related to the present invention, but several include features which resemble those of the present invention. Each has proven to be less than satisfactory in its own way, when used in an IV catheter or for irrigation. The present invention has been developed for the purpose of addressing and resolving these disadvantages.

Hypodermic syringe splatter shield, U.S. Pat. No. 4,898,588 (filed Oct. 17, 1988), provides a splatter shield for use with a hypodermic syringe to prevent high-angle back-splatter, from syringe lavage, into the user's face. The splatter shield comprises a preferably circular sheet of rigid material which is preferably colorless and transparent, having a central tube which is attached thereto and projects on at least one side of said sheet sufficiently to have one end adapted to receive a standard syringe fitting and the other end adapted to receive a standard hypodermic needle.

Prefilled catheter tip syringe kit, U.S. Pat. No. 4,954,239 (filed Apr. 15, 1988), provides a device for use as a single use, disposable, sterile irrigation kit for catheter irrigation, comprising a prefilled sterile piston syringe, including a catheter tip sized to operably connect to a catheter tube, and a removable cap which selectively prevents escape of fluid from the syringe and prevents movement of the piston while the tip is capped.

Syringe, U.S. Pat. No. 4,030,498 (filed Oct. 25, 1974), provides a contamination resistant syringe in which two sealing rings are axially spaced from each other. The sealing rings are prevented from sweeping the same area and there is no communication between the chamber defined by the two sealing rings and the discharge area forward of the inner sealing ring. Thus contamination which might enter from the rear of the barrel is prevented from contact with the inner sealing ring.

Safety syringe with retractable self-biased needle, U.S. Pat. No. 5,232,458 (filed Sep. 28, 1992), provides a safety syringe which includes a hollow needle secured in a front portion of the syringe for injection use, having a needle head portion formed on a rear portion of the needle, and a plunger slidably held in the syringe for boosting a liquid medicine in the syringe to be injected into a patient through the hollow needle.

Method and apparatus for dealing with intravenous fluids, U.S. Pat. No. 4,838,875 (filed Dec. 9, 1987), provides an apparatus for intravenous administration of fluid to which fluid may be added according to the process also disclosed, by injecting same, using a syringe, but not a needle, directly into the hollow portion of the apparatus through an opening therein. A normally closed backflow check valve device, having a component thereof acting as a means for receiving the fluid by engagement to the syringe, has been permanently sealed into the opening in the hollow portion of the apparatus. The apparatus is capped by a double luer locking cap, the purpose of which is to protect the opening into the apparatus from contamination.

Internal expansion syringe adaptor, U.S. Pat. No. 6,955,660 (filed Sep. 9, 2002), provides an adaptor for connecting a syringe to an aspiration or irrigation device, typically a medical device such as a catheter, cannula or needle, is described herein. The adaptor is comprised of two components: a deformable elastomeric expansion sleeve and a relatively rigid insertion body.

Syringe safety sleeve and adaptor, U.S. Pat. No. 6,287,282 (filed Nov. 16, 1999), provides a protective sleeve and adaptor hub combination for retrofitting conventional medical syringes, so that the syringe needle may be completely enclosed in the protective sleeve following use of the syringe. Once connected, the hub and sleeve cannot be removed from the syringe.

Adaptor system for use with a syringe, U.S. Pat. No. 5,609,584 (filed May 18, 1994), provides an adaptor system for a syringe that has a fluid carrying body. In a preferred embodiment, the adaptor system comprises a syringe fitting that has a hollow element protruding therefrom, and is attachable to the syringe such that the hollow element is in fluid communication with the fluid carrying body. The system also includes an adaptor member that has a port adapted to receive the hollow element of the syringe fitting in fluid-tight engagement.

Most of the prior art syringes and syringe adapters focus on certain problems which are unique to the injection of fluids directly into the patient's body from the syringe. These do not effectively address the issue of contamination of the fluid in the syringe when a significant amount of time elapses between drawing the fluid from a bottle, and either injecting it into a catheter, or using it for irrigation. Those prior art inventions which do address this issue have proven to be unsatisfactory. A syringe adaptor, which is attached to the syringe before introducing the fluid into the patient's body and prevents contamination of the fluid therein, would resolve these problems.

### SUMMARY OF THE INVENTION

Accordingly, the invention is directed to a syringe adaptor. After a withdrawal of sterile fluid in a syringe from a prescription bottle, the adaptor may be attached to the syringe to prevent contamination of the fluid. The fluid may then be safely injected into the patient's body or used for bladder irrigation.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWING

The accompanying drawing is included to provide a further understanding of the invention and is incorporated into and constitutes a part of the specification. It illustrates one embodiment of the invention and, together with the description, serves to explain the principles of the invention.

FIG. 1 shows a side perspective view of the first exemplary embodiment, displaying the adaptor 10, and the plunger 11.

FIG. 2 shows a side perspective view of a syringe, displaying the syringe 12, the needle 13, and the barrel 14.

FIG. 3 shows a sider view of the first exemplary embodiment, displaying the adaptor 10 and the plunger 11 as well as the syringe 12 within the adaptor 10.

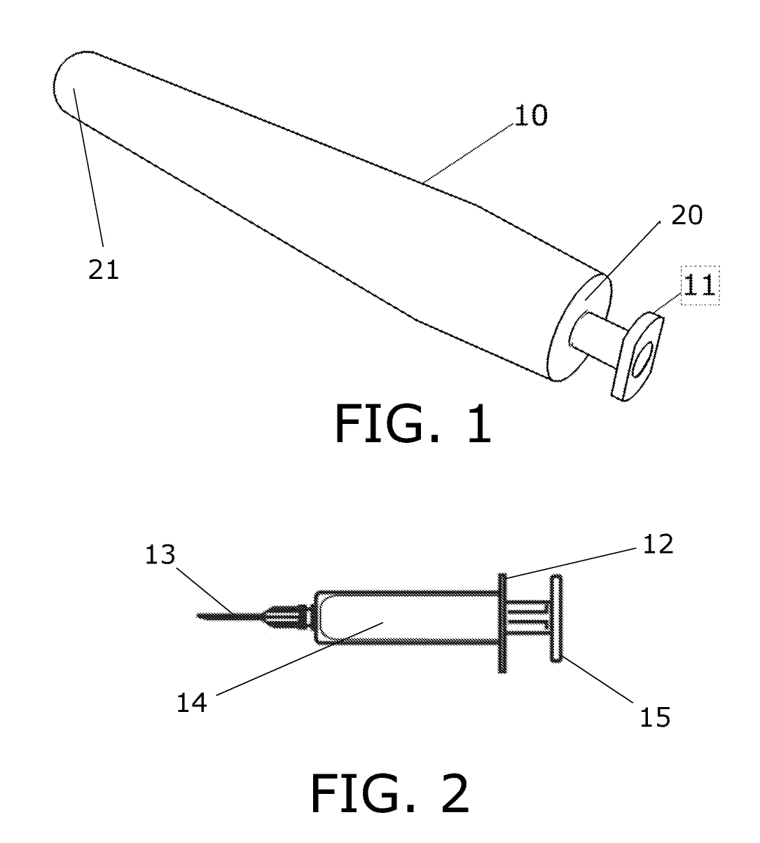

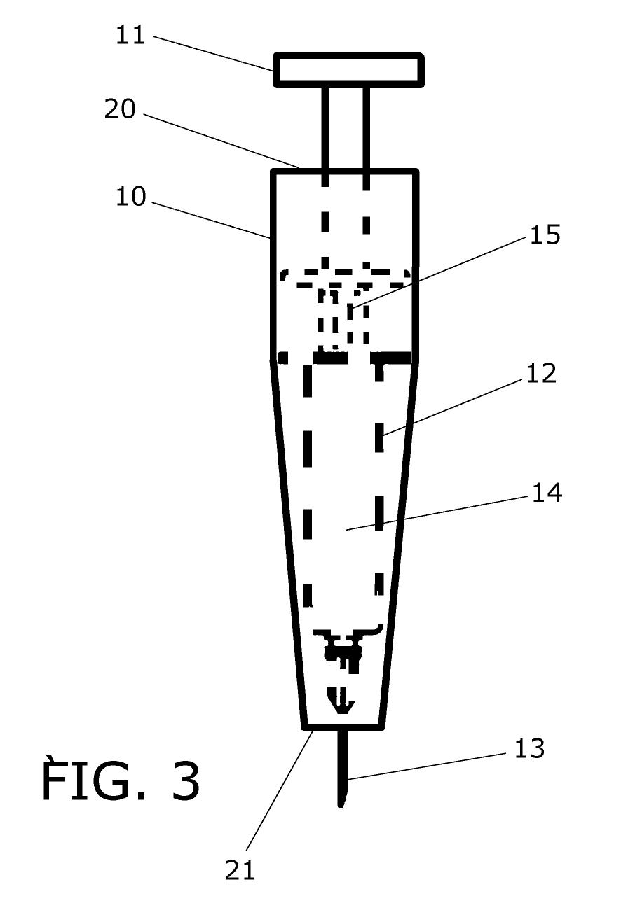

### DETAILED DESCRIPTION OF THE INVENTION

Referring now to the invention in more detail, the invention is directed to a syringe adaptor 10.

The first exemplary embodiment is comprised of a hollow cylindrical syringe adaptor 10, which provides an adapter plunger 11 at the upper end 20 of the adaptor 10. The adaptor 10 fits tightly over the barrel 14 of the syringe 12, such that the needle 13 of the syringe 12 emerges from the lower end 21 of the adaptor 10, and the adapter plunger 11 operates the plunger of the syringe 15.

To use the first exemplary embodiment, the user fills the syringe 12, attaches the adaptor 10 to the syringe 12, inserts the needle 13 of the syringe 12 into the catheter, and depresses the plunger 11 to transfer the fluid from the syringe 12 into the catheter.

The adaptor 10 is preferably manufactured from a rigid, disposable material which is capable of sterilization, such as plastic or acrylic polymer. Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A syringe adapter, comprising:
a. a hollow syringe adapter having an upper end and a lower end;
b. an adapter plunger;
c. said adapter plunger being provided at said upper end of said hollow cylindrical syringe adapter;
d. said syringe adapter being of a size and shape such that a syringe fits snuggly within said syringe adapter;
wherein, when said syringe is placed within said syringe adapter, said syringe adapter seals said syringe within said syringe adapter; said adapter plunger is configured to depress a syringe plunger when said adapter plunger is depressed;
and a needle of said syringe emerges from said lower end of said syringe adapter.

2. The syringe adapter of claim 1, wherein said syringe adapter is cylindrical in shape.

3. The syringe adapter of claim 1, wherein said syringe adapter is made from a rigid disposable material which is capable of sterilization.

4. The syringe adapter of claim 3, wherein said rigid disposable material is plastic.

5. The syringe adapter of claim 3, wherein said rigid disposable material is acrylic polymer.
